(19) United States
(12) Patent Application Publication
(10) Pub. No.: US 2010/0285126 A1
(43) Pub. Date: Nov. 11, 2010

(54) PHARMACEUTICAL COMPOSITIONS OF FENOFIBRATE

(76) Inventors: Rahul Dabre, Nagpur (IN); Roshanlal Sandal, Ferozpur (IN); Vikrant Thakkar, Sampat (IN); Girish Kumar Jain, Delhi (IN)

Correspondence Address: BIO INTELLECTUAL PROPERTY SERVICES (BIO IPS) LLC, 8509 KERNON CT., LORTON, VA, 22079, US

(21) Appl. No.: 12/671,832

(22) PCT Filed: Aug. 1, 2008
(86) PCT No.: PCT/IB08/53091

§ 371 Date: Jul. 28, 2010

(30) Foreign Application Priority Data
Aug. 2, 2007 (IN) 1384/MUM/2007
Aug. 2, 2007 (IN) 1485/MUM/2007
Aug. 2, 2007 (IN) 1486/MUM/2007
Aug. 2, 2007 (IN) 1487/MUM/2007
Aug. 2, 2007 (IN) 1488/MUM/2007
Mar. 24, 2008 (IN) 600/MUM/2008

(51) Int. Cl. A61K 9/28; A61K 31/216; A61P 7/00; A61P 3/06

(52) U.S. Cl. 424/474; 514/543

(57) ABSTRACT

The invention relates to pharmaceutical compositions comprising non-micronized fenofibrate with one or more pharmaceutically acceptable vehicles. The invention also relates to pharmaceutical compositions comprising non-micronized fenofibrate with one or more cyclodextrin derivatives. The invention also relates to processes for the preparation of such compositions.

### FIELD OF THE INVENTION

The invention relates to pharmaceutical compositions comprising non-micronized fenofibrate with one or more pharmaceutically acceptable vehicles. The invention also relates to pharmaceutical compositions comprising non-micronized fenofibrate with one or more cyclodextrin derivatives. The invention also relates to processes for the preparation of such compositions.

### BACKGROUND OF THE INVENTION

Fenofibrate is a lipid-regulating agent and belongs to the family of fibrates or fibric acid derivatives. It is indicated as an adjunctive therapy to diet for the treatment for adult patients with very high elevations of serum triglyceride levels who are at risk of pancreatitis and who do not respond adequately to dietary control. It is particularly useful for the treatment of adult endogenous hyperlipidemia, hypercholesterolemia and hypertriglyceridemia. It is commercially available as oral capsules containing micronized fenofibrate in the strengths of 67 mg, 134 mg and 200 mg

Fenofibrate is practically insoluble in water and exhibits a low rate of dissolution in aqueous media that results in inadequate bioavailability after oral ingestion. This low rate of dissolution of fenofibrate in aqueous media is also found in gastrointestinal fluids. Chemically, fenofibrate is 2-[4-(4-Chlorobenzoyl)phenoxy]-2-methylpropanoic acid 1-methylethyl ester of Formula I. Several methods of increasing the rate of dissolution of drugs having low solubility in water and other aqueous media have been disclosed in the prior art.

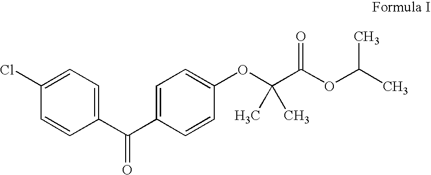

U.S. Pat. Nos. 5,145,684; 6,375,986; 6,969,529; and 6,592,903 disclose nanoparticulate compositions of fenofibrate.

U.S. Pat. Nos. 6,277,405; 6,652,881; 7,037,529; 7,041,319; 6,589,552; 6,531,158 and U.S. Patent Application Nos. 20040057998; 20040058005 and 2004137055 disclose micronized fenofibrate compositions.

U.S. Pat. Nos. 4,895,726; 5,880,148 and U.S. Application No. 20040071771 describe co-micronizing the fenofibrate with surface-active agents.

U.S. Pat. No. 6,555,135 describes co-micronized mixture of fenofibrate with pharmaceutically acceptable excipient that is not a surfactant.

U.S. Pat. Nos. 6,074,670 and 6,277,405 disclose micronized fenofibrate coated onto hydro soluble carriers with optional surface-active agents.

U.S. Pat. No. 6,828,334 describes inclusion complex of fenofibrate with cyclodextrins.

U.S. Pat. No. 6,027,747 describes solid dispersion of fenofibrate.

U.S. Patent Application No. 20040087656 describes fenofibrate of particle size less than 2000 nm with an improved bioavailability.

U.S. Patent Application Nos. 20060222706 and 20060222707 describe fenofibrate in intimate association with menthol or surfactant mixture.

U.S. Patent Application No. 20030138496 micronized fenofibrate with inert hydro soluble carriers.

Several other patents and applications describe specific formulations of micronized fenofibrate with specific polymeric or surface-active agent additives while several others describe emulsion and suspension formulations of fenofibrate.

The solubility of an active pharmaceutical ingredient influences the bioavailability of the drug. Fenofibrate is a poorly soluble drug. Due to its poor hydrosolubility, fenofibrate poses problem of low dissolution. It is also poorly absorbed in the digestive tract and consequently its bioavailability is incomplete and irregular. Clearly, there is a need for improved compositions in which the fenofibrate exhibits better dissolution properties.

### SUMMARY OF THE INVENTION

In one general aspect there is provided a pharmaceutical composition of fenofibrate or salts thereof. The composition includes non-micronized fenofibrate and one or more pharmaceutically acceptable vehicles.

In another general aspect there is provided a pharmaceutical composition of fenofibrate or salts thereof. The composition includes non-micronized fenofibrate, polyethylene glycol or derivatives thereof optionally, with one or more pharmaceutically acceptable excipients.

In another general aspect there is provided a process for preparing a pharmaceutical composition of fenofibrate or salts thereof. The process includes the steps of melting, mixing one or more pharmaceutically acceptable vehicles, non-micronized fenofibrate optionally, with one or more pharmaceutically acceptable excipients.

In another general aspect there is provided a pharmaceutical composition of fenofibrate or salts thereof. The composition includes non-micronized fenofibrate, cyclodextrin or a derivative thereof optionally, with one or more pharmaceutically acceptable excipients.

In another general aspect there is provided a process for preparing a pharmaceutical composition of fenofibrate or salts thereof. The process includes: a) melting one or more pharmaceutically acceptable vehicles to form a clear solution; b) dissolving non-micronized fenofibrate in the clear solution; c) spraying the fenofibrate solution onto one or more pharmaceutically acceptable excipients; and d) mixing with one or more other pharmaceutically acceptable excipients and converting it into a suitable dosage form.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

The details of one or more embodiments of the inventions are set forth in the description below. Other features, objects and advantages of the inventions will be apparent from the description and claims.

### DETAILED DESCRIPTION OF THE INVENTION

Fenofibrate is practically insoluble in water. This insolubility characteristic causes fenofibrate to exhibit a low rate of dissolution in aqueous media, e, g., gastrointestinal fluids, which results in inadequate bioavailability after oral ingestion. The present inventors while working on the fenofibrate formulation have found that when fenofibrate is dissolved in a vehicle at a specific temperature, and further processed with pharmaceutically acceptable excipients, the crystallinity of the fenofibrate is significantly reduced in the product. The reduction in crystallinity significantly enhances the solubility, dissolution and bioavailability of fenofibrate. The present inventors also found that when a non-micronized fenofibrate is complexed with cyclodextrin or a derivative thereof, the solubility of the mix so obtained is increased in the aqueous fluids, which in turn leads to a significant increase in percent release of the drug fenofibrate and hence increase in bioavailability.

The term ‘non-micronized fenofibrate’ as used herein refers to fenofibrate having a particle size greater than or equal to about 50 μm and fenofibrate is not subjected to any comminution techniques, such as milling, spray drying, high pressure homogenization, and the like known to a person skilled in the art.

Suitable vehicles which can be used in the process of the present invention are known to a person having ordinary skills in the art. Examples of such vehicles include polyethylene glycol or derivatives thereof, poloxamer, cremophore RH 40, vitamin E TPGS, and the like. Mixtures of vehicles are also suitable.

Suitable polyethylene glycol (PEG) or derivatives thereof may include all PEGs that are liquid at room temperature or that melt up to about 70° C. Suitable polyethylene glycol or derivatives thereof may include one or more of PEG 200, PEG 300, PEG 400, PEG 600, PEG 1000, PEG 4000, PEG 6000, PEG 8000, PEG 20000, polyglycolized glycerides, polyethylene glycol-polyoxyethylene, polyethylene glycol polypropylenes, polyethylene glycol-polyoxypropylene, and the like.

The composition of the present invention may be prepared by dissolving non-micronized fenofibrate in a clear solution of one or more melted vehicles. The obtained solution may be sprayed onto suitable filler. The obtained mixture may be granulated, with one or more pharmaceutically acceptable excipients. The obtained granules may be optionally coated and optionally mixed with one or more pharmaceutically acceptable excipients. The resulting mixture may be filled into capsules or compressed to make tablets.

Suitable pharmaceutically acceptable sugars may include one or more of sucrose, glucose, fructose, galactose, maltose, isomaltose, cellobiose, melibiose, gentiobiose, lactose, sorbitol, mannitol, and the like.

Suitable surfactants may include one or more of amphoteric, non-ionic, cationic or anionic surfactants. Examples of such surfactants include sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil polyoxyethylene fatty acid glycerides, poloxamer, cremophore RH 40, and the like. Mixtures of surfactants may also be used.

Suitable cyclodextrin or derivatives thereof may include one or more of hydroxypropyl-β-cyclodextrin, β-cyclodextrin, α-cyclodextrin, hydroxypropyl-α-cyclodextrin, and the like.

The composition comprising non-micronized fenofibrate with cyclodextrin or derivatives thereof may be prepared by the processes known in the art. The inclusion complex of a portion of total non-micronized fenofibrate with β-cyclodextrin may be prepared by dissolving fenofibrate and cyclodextrin or a derivative thereof in a suitable solvent followed by mixing and drying. The inclusion complex so formed may be mixed with one or more pharmaceutically acceptable excipients.

The solvents may include one or more of water, methanol, ethanol, isopropanol, acetone, ether, chloroform, dimethylsulfoxide, dimethylformamide, methylene chloride, and the like.

The melt granulation may be carried out by dissolving the remaining portion in part or full of the non-micronized fenofibrate in one or more pharmaceutically acceptable carriers by melting. The obtained melt may be mixed with a solution of a pharmaceutically acceptable sugar and one or more pharmaceutically acceptable excipients to get a molten mixture. The obtained mixture may be granulated by adsorbing it on one or more pharmaceutically acceptable excipients.

The term ‘portion’ as used herein refers to from about 1% to about 99% of the total non-micronized fenofibrate used in the formulation that needs to be complexed with cyclodextrin or a derivative thereof.

The term ‘remaining portion’ as used herein refers to from about 1% to about 99% of the total non-micronized fenofibrate used in the formulation that needs to be melt granulated with one or more pharmaceutically acceptable carriers.

The inclusion complex and granules obtained by melt granulation may be mixed together and may be optionally mixed with one or more pharmaceutically acceptable excipients. The resulting mixture may be filled into capsules or compressed to make tablets.

The one or more pharmaceutically acceptable excipients may include one or more of fillers, binders, lubricants, sweeteners, coloring and flavoring agents, glidants, disintegrants, and the like.

Suitable fillers may be one or more of microcrystalline cellulose, silicified micro-crystalline cellulose, mannitol, calcium phosphate, calcium sulfate, kaolin, dry starch, powdered sugar, and the like.

Suitable binders may be one or more of povidone, starch, stearic acid, gums, hydroxypropylmethyl cellulose, and the like.

Suitable lubricants may be one or more of magnesium stearate, zinc stearate, calcium stearate, stearic acid, sodium stearyl fumarate, hydrogenated vegetable oil, glyceryl behenate, and the like.

Suitable glidants may be one or more of colloidal silicon dioxide, talc, cornstarch, and the like.

Suitable disintegrants may be one or more of starch, croscarmellose sodium, crospovidone, sodium starch glycolate, and the like.

The pharmaceutical composition of the invention may be present in the form of a tablet, capsule, powder, disc, caplet, granules, pellets, tablet in tablet, tablet in capsule, pellets in capsule, powder in capsule, granules in capsule and other dosage forms suitable for oral administration. The tablets may further be coated with film forming polymers.

Examples of some film forming polymers that can be used for the coating include but are not limited to cellulose derivatives (hydroxypropyl methylcellulose, hydroxyethyl-cellulose, hydroxypropyl cellulose and their derivatives), acrylic and methacrylic copolymers of different molecular weights, and mixtures thereof.

The coating layers over the tablet may be applied as a solution/dispersion of the coating ingredients using conventional techniques known in the art selected from spray coating in a conventional coating pan or a fluidized bed processor, dip coating, and the like.

The invention is further illustrated by the following examples which are provided merely to be exemplary of the invention and do not limit the scope of the invention. Certain modifications and equivalents will be apparent to those skilled in the art and are intended to be included within the scope of the invention.

### EXAMPLES

The composition of batches is provided in Table 1 to 13. Following formulations are representatives of the preferred compositions of the invention. The preparation of example is detailed below.

### Example 1

TABLE 1
Composition of Fenofibrate Tablets

| | | |
|---|---|---|
| SN | Ingredients | Qty/Tab (%) |

Part I

| | | |
|---|---|---|
| 1. | Fenofibrate | 10 to 70 |
| 2. | PEG 6000 | 5 to 70 |
| 3. | Vitamin E TPGS | 2 to 50 |
| 4. | Starch 1500/Lactose | 10 to 70 |

Part II Extragranular

| | | |
|---|---|---|
| 5. | Prosolv SMCC 90 | 10 to 50 |
| 6. | Crospovidone | 1 to 10 |
| 7. | Aerosil 200 | 1 to 5 |
| 8. | Magnesium stearate | 0.1 to 2 |
| | Average weight of Core Tablet | |
| | Opadry | Weight gain 3.0% of core |

Procedure: A mixture of PEG 6000 and Vitamin E TPGS were melted together at 60-70° C. to form a clear solution. Fenofibrate was dissolved in the above solution at 60-70° C. (below the melting point of fenofibrate). Starch 1500/Lactose was loaded in a Fluid bed processor and the above solution of fenofibrate was sprayed onto starch 1500/Lactose. The temperature of the solution was maintained at 70-75° C. during spraying. The granules obtained in the above step were dried and passed through a suitable mesh screen and then were blended with presifted microcrystalline cellulose, Prosolv SMCC 50, Aerosil 200 and Crospovidone (Polyplasdone INF 10) for 10 min.

This blend was lubricated with pre-sifted magnesium stearate for 2-3 minutes. The lubricated blend was compresses into tablets using proposed tooling and then coated with aqueous dispersion of Opadry, to a 3% weight gain.

### Example 2

TABLE 2
Composition of Fenofibrate Tablets

| | | |
|---|---|---|
| SN | Ingredients | Workable range (%) |

Part I

| | | |
|---|---|---|
| 1. | Fenofibrate | 10 to 70 |
| 2. | PEG 6000 | 5 to 70 |
| 3. | Cremophore RH 40 | 2 to 50 |
| 4. | Starch 1500/Lactose/ | 10 to 70 |
| | Calcium Silicate | |

Part II Extragranular

| | | |
|---|---|---|
| 5. | Prosolv SMCC 90 | 10 to 50 |
| 6. | Crospovidone | 1 to 10 |
| 7. | Aerosil 200 | 1 to 5 |
| 8. | Magnesium stearate | 0.1 to 2 |
| | Average weight of Core Tablet | |
| | Opadry | Weight gain 3.0% of core |

Procedure: A mixture of PEG 6000 and Cremophore RH 40 was melted together at 60-70° C. to form a clear solution. Fenofibrate was dissolved in the above solution at 60-70° C. (below the melting point of fenofibrate). A mixture of starch 1500, lactose and calcium silicate was loaded in a Fluid bed processor and the above solution of fenofibrate was sprayed onto starch 1500, lactose and calcium silicate mixture. The temperature of the solution was maintained at 70-75° C. during spraying. The granules obtained in the above step were dried & passed through a suitable mesh screen and then were blended with pre-sifted microcrystalline cellulose. Prosolv SMCC 50, Aerosil 200 and Crospovidone (Polyplasdone INF 10) for 10 min. This blend was lubricated with pre-sifted magnesium stearate for 2-3 minutes. The lubricated blend was compressed into tablets using proposed tooling and then coated with aqueous dispersion of Opadry to a 3% weight gain.

### Example 3

TABLE 3
Composition of Fenofibrate Tablets

| | | |
|---|---|---|
| SN | Ingredients | Qty/Tab (%) |

Part I

| | | |
|---|---|---|
| 1. | Fenofibrate | 10 to 70 |
| 2. | PEG 6000 | 5 to 70 |
| 3. | Tween 80 | 2 to 50 |
| 4. | Starch 1500/Lactose/ | 10 to 70 |
| | Calcium Silicate | |

Part II Extragranular

| | | |
|---|---|---|
| 5. | Prosolv SMCC 90 | 10 to 50 |
| 6. | Crospovidone | 1 to 10 |
| 7. | Aerosil 200 | 1 to 5 |
| 8. | Magnesium stearate | 0.1 to 2 |
| | Average weight of Core Tablet | |
| | Opadry | Weight gain 3.0% of core |

Procedure: A mixture of PEG 6000+Tween 80 were melted together at 60-70° C. to form a clear solution. Fenofibrate was dissolved in the above solution at 60-70° C. (below the melting point of fenofibrate). A mixture of starch 1500, lactose and calcium silicate was loaded in a Fluid bed processor and the above solution of fenofibrate was sprayed onto starch 1500, lactose and calcium silicate mixture. The temperature of the solution was maintained at 70-75° C. during spraying. The granules obtained in the above step were dried & passed through a suitable mesh screen and then were blended with pre-sifted, microcrystalline cellulose, Prosolv SMCC 50, Aerosil 200 and Crospovidone (Polyplasdone INF 10) for 10 min. This blend was lubricated with a pre-sifted magnesium stearate for 2-3 minutes. The lubricated blend was compressed into tablets using proposed tooling and then coated with aqueous dispersion of Opadry to a 3% weight gain.

### Example 4

TABLE 4
Composition of Fenofibrate Tablets

| | | |
|---|---|---|
| SN | Ingredients | Qty/Tab (%) |

Part I

| | | |
|---|---|---|
| 1. | Fenofibrate | 10 to 70 |
| 2. | Poloxamer 407 or 188 | 5 to 70 |
| 3. | Vitamin E TPGS | 2 to 50 |
| 4. | Starch 1500/Lactose | 10 to 70 |

Part II Extragranular

| | | |
|---|---|---|
| 5. | Prosolv SMCC 90 | 10 to 50 |
| 6. | Crospovidone | 1 to 10 |
| 7. | Aerosil 200 | 1 to 5 |
| 8. | Magnesium stearate | 0.1 to 2 |
| | Average weight of Core Tablet | |
| | Opadry | Weight gain 3.0% of core |

Procedure: A mixture of Poloxamer 407 or 188 and Vitamin E TPGS was melted together at 60-70° C. to form a clear solution. Fenofibrate was dissolved in the above solution at 60-70° C. (below the melting point of fenofibrate). Starch 1500/lactose was loaded in a Fluid bed processor and the above solution of fenofibrate was sprayed onto starch 1500/lactose. The temperature of the solution was maintained at 70-75° C. during spraying. The granules obtained in the above step were dried & passed through a suitable mesh screen and then were blended with pre-sifted microcrystalline cellulose, Prosolv SMCC, 50, Aerosil 200 and Crospovidone (Polyplasdone INF 10) for 10 min. This blend was lubricated with a pre-sifted magnesium stearate for 2-3 minutes. The lubricated blend was compressed into tablets using proposed tooling and then coated with aqueous dispersion of Opadry to a 3% weight gain.

### Example 5

TABLE 5
Composition of Fenofibrate Tablets

| | | |
|---|---|---|
| SN | Ingredients | Qty/Tab (%) |

Part I

| | | |
|---|---|---|
| 1 | Fenofibrate | 10 to 70 |
| 2 | Vitamin E TPGS | 2 to 50 |
| 3 | Starch 1500/Lactose | 10 to 70 |

Part II Extragranular

| | | |
|---|---|---|
| 4 | Prosolv SMCC 90 | 10 to 50 |
| 5 | Crospovidone | 1 to 10 |
| 6 | Aerosil 200 | 1 to 5 |
| 7 | Magnesium stearate | 0.1 to 2 |
| | Average weight of Core Tablet | |
| 8 | Opadry | Weight gain 3.0% of core |

Procedure: Vitamin E TPGS was melted at 60-70° C. to form a clear solution. Fenofibrate was dissolved in the above solution at 60-70° C. (below the melting point of fenofibrate). Starch 1500/lactose was loaded in a Fluid bed processor and the above solution of fenofibrate was sprayed onto starch 1500/lactose. The temperature of the solution was maintained at 70-75° C. during spraying. The granules obtained in the above step were dried & passed through a suitable mesh screen and then were blended with pre-sifted microcrystal line cellulose, Prosolv SMCC 50, Aerosil 200 and Crospovidone (Polyplasdone INF 10) for 10 min. This blend was lubricated with pre-sifted magnesium stearate for 2-3 minutes. The lubricated blend was compressed into tablets using proposed tooling and then coated with aqueous dispersion of Opadry to a 3% weight gain.

TABLE 6
Dissolution data of Fenofibrate tablets (145 mg)
and Tricor ® Tablets (145 mg)

| | % drug released | % drug released | % drug released |
|---|---|---|---|
| Time(min) | (Example-4) | (Example-1) | (Tricor ® tablets) |
| 10 | 28 | 30 | 37 |
| 20 | 52 | 56 | 85 |
| 30 | 70 | 76 | 92 |
| 45 | 92 | 96 | 94 |
| 60 | 101 | 104 | 95 |
| 90 | 103 | 107 | 96 |

Table 6 provides the dissolution data for fenofibrate tablets (145 mg) prepared as per the formula given in Table 4 and commercially available Tricor® Tablets. For determination of drug release rate, USP Type 2 Apparatus (rpm 50) was used, wherein 1000 ml of 0.025 M SLS in water at 37° C.±0.5° C. was used as a medium.

Table 7 provides the dissolution data for fenofibrate tablets (145 mg) prepared as per the formula given in Table 4 and commercially available Tricor® Tablets. For determination of drug release rate, USP Type 2 Apparatus (rpm 50) was used, wherein 2000 ml of 0.0125M SLS in water at 37° C.±0.5° C. was used as a medium.

TABLE 7
Dissolution data of Fenofibrate tablets (145 mg)
and Tricor ® Tablets (145 mg)

| | % drug released | % drug released | % drug released |
|---|---|---|---|
| Time(min) | (Example-4) | (Example-1) | (Tricor ® tablets) |
| 10 | 29 | 27 | 52 |
| 20 | 47 | 50 | 95 |
| 30 | 67 | 71 | 98 |
| 45 | 90 | 92 | 98 |
| 60 | 97 | 102 | 99 |
| 90 | 99 | 107 | 99 |

### Example 6

TABLE 8
Composition of Fenofibrate Tablets

| | | |
|---|---|---|
| S. No | Ingredients | Qty/tab (% w/w) |

Part I

| | | |
|---|---|---|
| 1 | Fenofibrate | 5-50 |
| 2 | PEG 6000 | 5-50 |

Part II

| | | |
|---|---|---|
| 3 | Sucrose | 0.5-20 |
| 4 | Povidone K-30 | 0.5-20 |

Part III

| | | |
|---|---|---|
| 5 | Lactose monohydrate | 5-40 |
| 6 | Crospovidone | 5-40 |
| 7 | Poloxamer | 5-40 |
| 8 | Sodium Lauryl Sulfate | 1-20 |
| 9 | Cremophore RH 40 | 5-40 |
| 10 | Docusate Sodium | 1-20 |

Part IV

| | | |
|---|---|---|
| 11 | Prosolv SMCC 90 | 1-25 |
| 12 | Crospovidone | 1-25 |
| 13 | Magnesium Stearate | 1-15 |

Procedure: Fenofibrate and PEG were melted in a water bath to get a molten mass. Sucrose and Povidone K-30 were dissolved in a suitable solvent and was added to the molten mass and homogenized. The mixture so obtained was adsorbed on lactose monohydrate, crospovidone, poloxamer, sodium lauryl sulfate, Cremophore RH 40 and docusate sodium in a Rapid Granulator Mixer to get uniform granules. The obtained granules were mixed with Prosolv SMCC 90 and crospovidone. The resulting blend was lubricated with magnesium stearate and compressed into tablets using suitable tooling. The tablets can be optionally coated with aqueous dispersion of Opadry.

### Example 7

TABLE 9
Composition of Fenofibrate Tablets

| | | |
|---|---|---|
| S. No | Ingredients | Qty/tab (% w/w) |

Part I

| | | |
|---|---|---|
| 1 | Fenofibrate | 5-50 |
| 2 | PEG 6000 | 5-50 |
| 3 | Sorbitol | 0.5-20 |
| 4 | Povidone K-30 | 0.5-20 |

Part II

| | | |
|---|---|---|
| 5 | Lactose monohydrate | 5-40 |
| 6 | Crospovidone | 5-40 |
| 7 | Poloxamer | 5-40 |
| 8 | Sodium Lauryl Sulfate | 1-20 |
| 9 | Cremophore RH 40 | 5-40 |
| 10 | Docusate Sodium | 1-20 |

Part III

| | | |
|---|---|---|
| 11 | Prosolv SMCC 90 | 1-25 |
| 12 | Crospovidone | 1-25 |
| 13 | Magnesium Stearate | 1-15 |

Procedure: Fenofibrate, PEG 6000 and sorbitol were melted in a water bath to get a molten mass and a solution of Povidone K-30 was added to the molten mass and homogenized. The obtained molten mass was adsorbed on lactose monohydrate, crospovidone, poloxamer, sodium lauryl sulfate, Cremophore RH 40 and docusate sodium in a Rapid Granulator Mixer to get uniform granules. The obtained granules were mixed with Prosolv SMCC 90 and crospovidone. The resulting blend was lubricated with magnesium stearate and compressed into tablets using suitable tooling. The tablets can be optionally coated with aqueous dispersion of Opadry.

### Example 8

TABLE 10
Composition of Fenofibrate Tablets

| | | |
|---|---|---|
| S. No | Ingredients | Qty/tab (% w/w) |

Part I

| | | |
|---|---|---|
| 1 | Fenofibrate | 5-50 |
| 2 | PEG 6000 | 5-50 |
| 3 | Sorbitol | 0.5-20 |
| 4 | Povidone K-30 | 0.5-20 |

Part II

| | | |
|---|---|---|
| 5 | Lactose monohydrate | 5-40 |
| 6 | Crospovidone | 5-40 |

Part III

| | | |
|---|---|---|
| 7 | Poloxamer | 5-40 |
| 8 | Sodium Lauryl Sulfate | 1-20 |
| 9 | Cremophore RH 40 | 5-40 |
| 10 | Docusate Sodium | 1-20 |

Part IV

| | | |
|---|---|---|
| 11 | Prosolv SMCC 90 | 1-25 |
| 12 | Crospovidone | 1-25 |
| 13 | Magnesium Stearate | 1-15 |

Procedure: Fenofibrate and beta cyclodextrin were dissolved in a suitable solvent and mixed followed by drying to make an inclusion complex. PEG 6000 and fenofibrate were melted in a water bath to get a molten mass. Sucrose and HPMC were dissolved in a suitable solvent and added to the molten mass. The obtained molten mass was adsorbed on lactose monohydrate, crospovidone, poloxamer, sodium lauryl sulfate and docusate sodium in a Rapid Granulator Mixer to get uniform granules. The obtained granules were mixed with the inclusion complex, Prosolv SMCC: 90 and crospovidone. The resulting blend was lubricated with magnesium stearate and compressed into tablets using proposed tooling. The tablets can be optionally coated with aqueous dispersion of Opacity.

### Example 10

TABLE 12
Composition of Fenofibrate Tablets

| | | |
|---|---|---|
| S. No | Ingredients | Qty/tab (% w/w) |

Part I

| | | |
|---|---|---|
| 1 | Fenofibrate | 5-50 |
| 2 | Beta Cyclodextrin | 5-60 |
| 3 | Prosolv SMCC 90 | 5-30 |
| 4 | Crospovidone | 0.5-5 |
| 5 | Magnesium Stearate | 0.5-5 |

Part II

| | | |
|---|---|---|
| 6 | Fenofibrate | 5-50 |
| 7 | PEG 6000 | 5-50 |

Part III

| | | |
|---|---|---|
| 8 | HPMC | 1-25 |
| 9 | Sucrose | 1-25 |

Part IV

| | | |
|---|---|---|
| 10 | Lactose monohydrate | 5-25 |
| 11 | Poloxamer | 1-25 |
| 12 | Sodium Lauryl Sulfate | 1-25 |
| 13 | Docusate Sodium | 1-25 |

Part V

| | | |
|---|---|---|
| 14 | Prosolv SMCC 90 | 1-20 |
| 15 | Crospovidone | 1-20 |
| 16 | Magnesium Stearate | 1-20 |

Procedure: Fenofibrate and beta cyclodextrin were dissolved in a suitable solvent and mixed followed by drying to make an inclusion complex. Prosolv SMCC 90 and crospovidone were sifted and added to the inclusion complex and the resulting mixture was lubricated with magnesium stearate to form a Premix (A). PEG 6001 and fenofibrate were melted in a water bath to get a molten mass. Sucrose and HPMC were dissolved in a suitable solvent and was added to the molten mass. The obtained mixture was adsorbed on lactose monohydrate, poloxamer, sodium lauryl sulfate and docusate sodium in a Rapid Granulator Mixer to get uniform granules (Premix B). The obtained granules were mixed with Premix (A), Prosolv SMCC 90 and crospovidone. The resulting blend was lubricated with magnesium stearate and compressed into tablets using proposed tooling. The tablets can be optionally coated with aqueous dispersion of Opadry.

### Example 11

TABLE 13
Composition of Fenofibrate Tablets

| | | |
|---|---|---|
| S. No | Ingredients | Qty/tab (% w/w) |

Part I

| | | |
|---|---|---|
| 1 | Fenofibrate | 5-50 |
| 2 | PEG 6000 | 5-50 |

Part II

| | | |
|---|---|---|
| 3 | Sucrose | 0.5-20 |
| 4 | Povidone K-30 | 0.5-20 |

Part III

| | | |
|---|---|---|
| 5 | Lactose monohydrate | 5-40 |

Part IV

| | | |
|---|---|---|
| 6 | Crospovidone | 5-40 |
| 7 | Poloxamer | 5-40 |
| 8 | Sodium Lauryl Sulfate | 1-20 |
| 9 | Cremophore RH 40 | 5-40 |
| 10 | Docusate Sodium | 1-20 |

Part V

| | | |
|---|---|---|
| 11 | Prosolv SMCC 90 | 1-25 |
| 12 | Crospovidone | 1-25 |
| 13 | Magnesium Stearate | 1-15 |

Procedure: Fenofibrate and PEG were melted in a water bath to get a molten mass. Sucrose and Povidone K-30 were dissolved in a suitable solvent and was added to the molten mass and homogenized. The obtained molten mass was adsorbed on lactose monohydrate in a Rapid Granulator Mixer to get uniform granules. The obtained granules were coated with a solution of crospovidone, poloxamer, sodium lauryl sulfate. Cremophore RH 40 and docusate sodium and dried. The coated granules were mixed with Prosolv SMCC 90 and crospovidone. The resulting blend was lubricated with magnesium stearate and compressed into tablets using suitable tooling. The tablets can be optionally coated with aqueous dispersion of Opadry.

While the invention has been described in terms of its specific embodiments, certain modifications and equivalents will be apparent to those skilled in the art and are intended to be included within the scope of the invention.

1. A pharmaceutical composition of fenofibrate or salts thereof comprising non-micronized fenofibrate and one or more pharmaceutically acceptable vehicles.

2. The pharmaceutical composition of claim 1, wherein the pharmaceutically acceptable vehicle comprises one or more of polyethylene glycol or derivatives thereof, poloxamer, Cremophore RH 40 and vitamin E.

3. The pharmaceutical composition of claim 1 further comprising one or more pharmaceutically acceptable excipients.

4. The pharmaceutical composition of claim 3, wherein the pharmaceutically acceptable excipients comprise one or more of binders, lubricants, disintegrants and glidants.

5. The pharmaceutical composition of claim 1, wherein the composition is in the form of a tablet, capsule, powder, disc, caplet, granules, pellets, tablet in tablet, tablet in capsule, pellets in capsule, powder in capsule and granules in capsule.

6. A pharmaceutical composition of fenofibrate or salts thereof comprising non-micronized fenofibrate, polyethylene glycol or derivatives thereof optionally, with one or more pharmaceutically acceptable excipients.

7. The pharmaceutical composition of claim 6, wherein the polyethylene glycol or derivatives comprise one or more of PEG 200, PEG 300, PEG 400, PEG 600, PEG 1000, PEG 4000, PEG 6000, PEG 8000, PEG 20000, polyglycolized glycerides, polyethylene glycol-polyoxyethylenes, polyethylene glycol polypropylenes and polyethylene glycol-polyoxypropylenes.

8. The pharmaceutical composition of claim 6 further comprising one or more pharmaceutically acceptable sugars.

9. The pharmaceutical composition of claim 8, wherein the pharmaceutically acceptable sugar comprises one or more of sucrose, glucose, fructose, galactose, maltose, isomaltose, cellobiose, melibiose, gentiobiose, lactose, sorbitol and mannitol.

10. (canceled)

11. The pharmaceutical composition of claim 6 further comprising one or more pharmaceutically acceptable surfactants.

12. The pharmaceutical composition of claim 11, wherein the surfactant comprises one or more of amphoteric, non-ionic, cationic or anionic surfactants.

13. The pharmaceutical composition of claim 6, wherein the pharmaceutically acceptable excipients comprise one or more of binders, lubricants, disintegrants and glidants.

14. The pharmaceutical composition of claim 6, wherein the composition is in the form of a tablet, capsule, powder, disc, caplet, granules, pellets, tablet in tablet, tablet in capsule, pellets in capsule, powder in capsule and granules in capsule.

15. A process for preparing pharmaceutical composition of fenofibrate or salts thereof, the process comprising: (a) melting one or more pharmaceutically acceptable vehicles to form a clear solution; (b) dissolving non-micronized fenofibrate in the clear solution; (c) spraying the fenofibrate solution onto one or more pharmaceutically acceptable excipients; and (d) mixing with one or more other pharmaceutically acceptable excipients and converting it into a suitable dosage form.

16-19. (canceled)

20. A pharmaceutical composition of fenofibrate or salts thereof comprising non-micronized fenofibrate, cyclodextrin or derivatives thereof optionally, with one or more pharmaceutically acceptable excipients.

21. The pharmaceutical composition of claim 20, wherein the cyclodextrin or derivatives comprise one or more of hydroxypropyl-β-cyclodextrin, β-cyclodextrin, α-cyclodextrin and hydroxypropyl-α-cyclodextrin.

22. (canceled)

23. The pharmaceutical composition of claim 20, wherein the pharmaceutically acceptable excipients comprise one or more of binders, lubricants, disintegrants and glidants.

24. The pharmaceutical composition of claim 20, wherein the composition is in the form of a tablet, capsule, powder, disc, caplet, granules, pellets, tablet in tablet, tablet in capsule, pellets in capsule, powder in capsule and granules in capsule.

25. A process for preparing a pharmaceutical composition of fenofibrate or salts thereof, the process comprising the steps of complexing; melt-granulating non-micronized fenofibrate with cyclodextrin or a derivative thereof; and optionally, mixing with other pharmaceutically acceptable excipients.

26. The process of claim 25, wherein the cyclodextrin or derivatives thereof comprise one or more of hydroxypropyl-β-cyclodextrin, β-cyclodextrin, α-cyclodextrin and hydroxypropyl-α-cyclodextrin.

27. (canceled)

28. The process of claim 25, wherein the pharmaceutically acceptable excipients comprise one or more of binders, lubricants, disintegrants and glidants.

29. The pharmaceutical composition of claim 25, wherein the composition is in the form of a tablet, capsule, powder, disc, caplet, granules, pellets, tablet in tablet, tablet in capsule, pellets in capsule, powder in capsule and granules in capsule.
